(19) United States
(12) Patent Application Publication
(10) Pub. No.: US 2023/0330236 A1
(43) Pub. Date: Oct. 19, 2023

(54) CERVIDAE DEER FAMILY ANTLER PET CHEW WITH HEALTH ADDITIVES AND METHOD OF USE AND MAKING

(71) Applicants: David EISENHUTH, State College, PA (US); Kristy HOUCK, Tyrone, PA (US)

(72) Inventors: David EISENHUTH, State College, PA (US); Kristy HOUCK, Tyrone, PA (US)

(21) Appl. No.: 18/136,391

(22) Filed: Apr. 19, 2023

Related U.S. Application Data
(60) Provisional application No. 63/332,339, filed on Apr. 19, 2022.

(51) Int. Cl. A61K 47/46; A61K 9/00; A61K 35/60; A61K 31/201; A61K 35/742; A61K 36/064; A61K 36/8962; A61K 36/288; A61K 36/53; A61K 36/534; A61K 36/484; A61K 36/05; A61K 35/741; A61K 31/07; A61K 31/51; A61K 31/525; A61K 31/455; A61K 31/197; A61K 31/4415; A61K 31/375; A61K 31/593; A61K 31/355; A61K 31/122; A61K 31/14; A61K 33/06; A61K 33/26; A61K 33/42; A61K 33/00; A61K 33/32; A61K 33/24; A61K 33/30; A61K 33/34; A61K 33/04; A23K 10/26; A23K 20/174; A23K 20/24; A23K 20/20; A23K 20/26; A23K 20/158; A23K 20/105; A23K 10/18; A23K 20/163; A23K 10/30; A23K 20/189; A23K 50/40

(52) U.S. Cl. CPC A61K 47/46; A61K 9/0056; A61K 35/60; A61K 31/201; A61K 35/742; A61K 36/064; A61K 36/8962; A61K 36/288; A61K 36/53; A61K 36/534; A61K 36/484; A61K 36/05; A61K 35/741; A61K 31/07; A61K 31/51; A61K 31/525; A61K 31/455; A61K 31/197; A61K 31/4415; A61K 31/375; A61K 31/593; A61K 31/355; A61K 31/122; A61K 31/14; A61K 33/06; A61K 33/26; A61K 33/42; A61K 33/00; A61K 33/32; A61K 33/24; A61K 33/30; A61K 33/34; A61K 33/04; A23K 10/26; A23K 20/174; A23K 20/24; A23K 20/30; A23K 20/26; A23K 20/158; A23K 20/105; A23K 10/18; A23K 20/163; A23K 10/30; A23K 20/189; A23K 50/40

(57) ABSTRACT

A deer family antler pet chew includes one or more additives present in an effective amount in the deer family antler to improve at least the health of a dog ingesting the deer family antler dog chew. The one or more additives can be added to the dog chew using an infusion technique.

### FIELD OF THE INVENTION

The invention relates to a pet chew, and more particularly to a Cervidae deer family antler that is modified with the addition of health additives like minerals, supplements, vitamins, pre- and pro-biotics, and the like to provide not only a chewing function for a pet, e.g., a dog, but a therapeutic and health benefit function as well.

### BACKGROUND ART

In the art, it is known to make artificial or synthetic dog chews that are infused with various vitamins, minerals, and supplements. Activpetz Duck Jeryky Multivitamin & Immune Maintenance Formula—Shop LP (lovingpetsproducts.com) is an example of a man-made pet treat that includes vitamins, e.g., Vitamins A, B2, B6, B12, C, D3 and E, pre- and pro-biotics, glucosamine and chondroitin, omega-3 fish oils, linoleic acid, and the like as part of the treat to improve a pet's health and lifestyle.

It is also known to use deciduous deer family antlers from a deer as a dog chew. Antlers are unique to the deer family, and grow on more than sixty kinds of deer. They are made up of a bone-like substance, which originates as cartilage and slowly hardens. Antlers are shed every year, and a whole new one grows for the breeding season. During that time, they are very soft, flowing with blood and covered in a soft layer called velvet. Towards the end of their growth, the blood flow stops and the antler hardens, ready to impress females and fight off competing males.

Even though antlers are naturally shed annually, most that are used for pet treats are by-products of the hunting industry. This way, none of the hunted animal goes to waste. Once harvested, the antlers are cut down to size and may be split. The sharp points are removed or sanded down. They may then be washed or sanitized. There are no other modifications made to antlers for dog chews.

However, there is still a need to improve the health and/or lifestyle of pets in terms of pet treats or chews. The present invention responds to this need by providing natural deer antlers that are modified with health additives that are known to be beneficial to pets such as dogs.

### SUMMARY OF THE INVENTION

The invention is an improvement in Cervidae deer family antler chews for pets, particularly dogs, in that at least the inner portion of the deer family antler is supplemented with one or more health additives that are known to improve the health benefit to the pet chewing on the antler.

The invention also includes a method of improving the health of a pet, e.g., a dog, by providing a supplemental natural chew for the pet, wherein the chew is the inventive deer family antler pet chew.

The inventive deer family antler pet chew includes one or more additives that are present in an effective amount in the deer family antler chew to improve at least the health of a pet ingesting the deer family antler pet chew.

While any additive known to improve the health of a pet like a dog can be used in the inventive pet chew, examples of additives include vitamins, minerals, supplements for joints, for example, glucosamine and chondroitin, omega-3, -6 and -9 fish oils, linoleic acid, probiotics and prebiotics, for example, Fructooligosaccharide and Bacillus Coagulans with respective amounts for these additives being 50 mg/chew and 1 billion CFU/chew, natural ingredients as one or more of kelp, alfalfa herb, flaxseed meal, brewer's yeast, lecithin, spinach, garlic, chamomile, oatstraw, dandelion root, thyme, rosemary, peppermint, fennel, rose hips, licorice, ginger, barley grass, wheat grass, Irish moss, chlorella and natural spirulina, digestive enzymes, and anti-oxidants. Examples of vitamins includes Vitamin A, B vitamins, e.g., biotin, folate, niacin, pantothenic acid, riboflavin, thiamine, vitamin B-6, and vitamin B-12), Vitamin C, Vitamin D, Vitamin E, Vitamin K, and Choline. Examples of minerals includes Calcium, Magnesium, Iron, Phosphorus, Potassium, Manganese, Chromium, Zinc, Copper, and Selenium.

Preferably, the one or more additives are contained in at least the lamellar bone of the deer family antler.

The invention also entails an improved method of providing a pet chew to a pet, wherein the inventive chew is used for the pet.

While the one or more additives can be added to the deer family antler in any number of ways providing that the one or more additives are retained in the deer family antler so as to provide a benefit to the pet when the chew is ingested, a preferred method uses an infusion technique. In this method, the chew and a solution are provided, the solution containing the one or more additives. The deer family antler pet chew is placed in the solution so that the one or more additives are retained in at least the lamellar bone of the deer family antler so as to provide a health benefit to a pet using the deer family antler pet chew.

The solution can be maintained at ambient temperature or heated to accelerate the infusion process, wherein the heating is such that it does not adversely affect the antler or any of the additives contained in the solution.

### BRIEF DESCRIPTION OF THE DRAWINGS

FIG. 1 shows an example of the inventive deer antler pet chew.

FIGS. 2A-2D shows other examples of the inventive deer antler pet chew.

### DETAILED DESCRIPTION OF THE INVENTION

The invention provides an improved chew for a pet, wherein the chew is a natural deer family antler that has been modified by the addition or one or more health additives. The inventive chew described below is described in connection with a dog. However, the inventive chew can be used by any pet that would derive a benefit from the additives contained in the deer family antler chew. Examples of these pets that are not dogs include chinchilla, guinea pigs, rabbits, squirrels, hamsters, and bunnies.

Health additives include any additive that is recognized as being beneficial for the health of the dog. Examples of these additives are vitamins, e.g., A, B2, B6, B12, C, D3 and E, minerals, e.g., Calcium, Magnesium, Iron, Phosphorus, Potassium, Manganese, Chromium, Zinc, Copper, and Selenium, supplements for joints like glucosamine and chondroitin, omega-3, -6 and -9 fish oils, linoleic acid, and probiotics and prebiotics, e.g., Fructooligosaccharide and Bacillus Coagulans and respective amounts for these additives can be 50 mg/chew and 1 billion CFU/chew. Natural ingredients can be one or more of additive, e.g., kelp, alfalfa herb, flaxseed meal, brewer's yeast, lecithin, spinach, garlic, chamomile, oatstraw, dandelion root, thyme, rosemary, peppermint, fennel, rose hips, licorice, ginger, barley grass, wheat grass, irish moss, chlorella and natural spirulina. Digestive enzymes and anti-oxidants can also be used.

A preferred way to modify the deer family antler according to the invention is by infusing the one or more of the additives such that the additives are retained in at least the lamellar bone and associated marrows spaces within the deer family antler hard outer shell. This infusing process can be simply providing a solution having the desired concentration of the one or more additives dissolved in the solution and combining the deer family antler with the solution over a period of time so that the additives in the solution can infuse into at least the lamellar bone of the deer family antler. The additive-containing solution with the deer family antler therein could be used at room temperature, or heated, or pressurized to accelerate the infusion process but without degrading any of the additives in the solution. When using the infusion technique, the one or more additives should be capable of going into solution for infusion into the deer family antler.

The amount of the one or more additives is considered to be an effective amount that provides at least a health benefit to the dog. For example, amounts of glucosamine and chondroitin used for dog joint health are well known and the process for including such additives to the deer family antler could be designed such that the required amount is contained in the deer family antler. As noted above, examples of amounts of pre-and pro-biotics are shown above. Likewise, effective amounts of fish oils, vitamins, or other known healthy additives are all well known in the prior art for dogs and a further description of the known amounts for the health additives for dogs is not necessary for understanding of the invention.

While an infusion method is preferred to include the health additives to a deer family antler, any other known methods could be employed to modify the deer family antler such that it contains the one or more health additives that would be beneficial to a dog from at least a health standpoint.

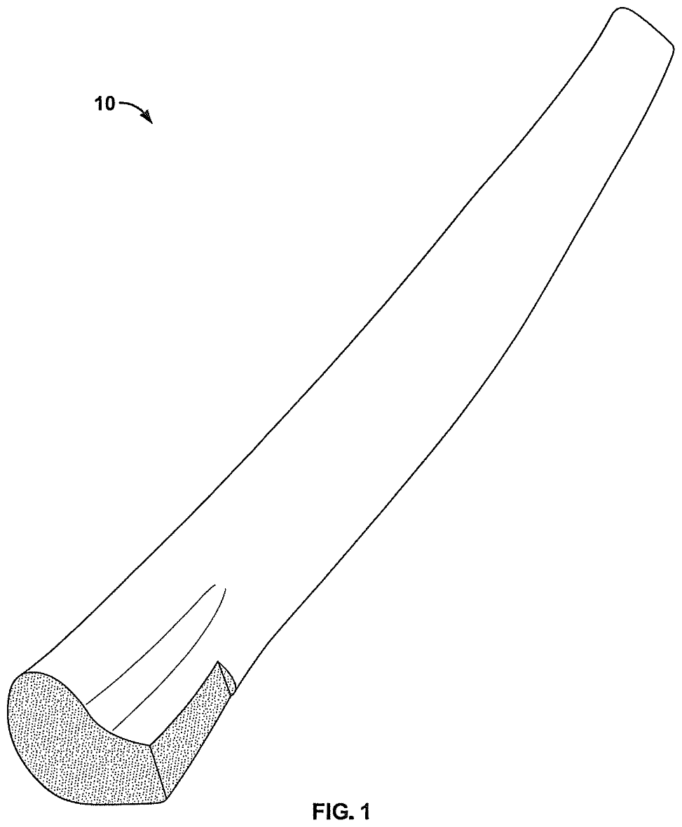

FIG. 1 illustrates an exemplary deer family antler 10 that is appropriately cut to length for use as a chew and modified with the one or more additives for dog health improvement. The deer family antler shown in the FIG. 1 is just one configuration of an antler and other configurations can be used as the inventive chew, e.g., a split antler.

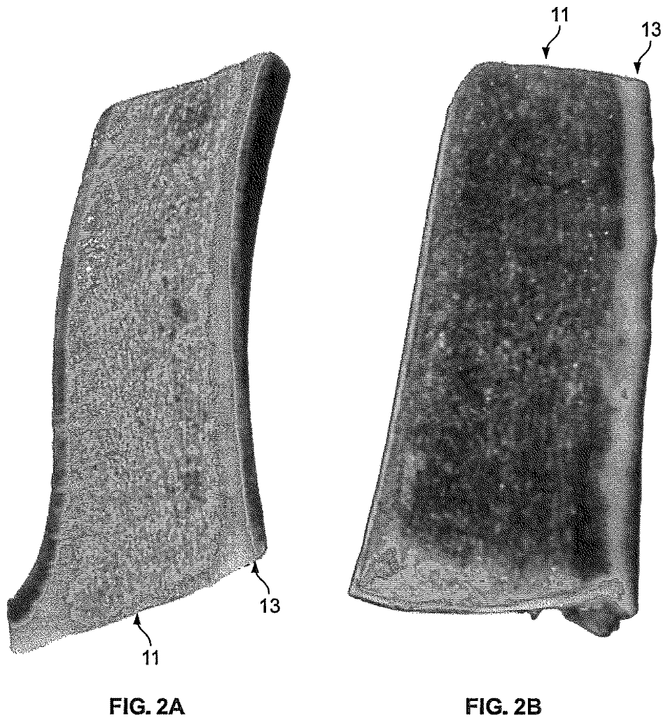

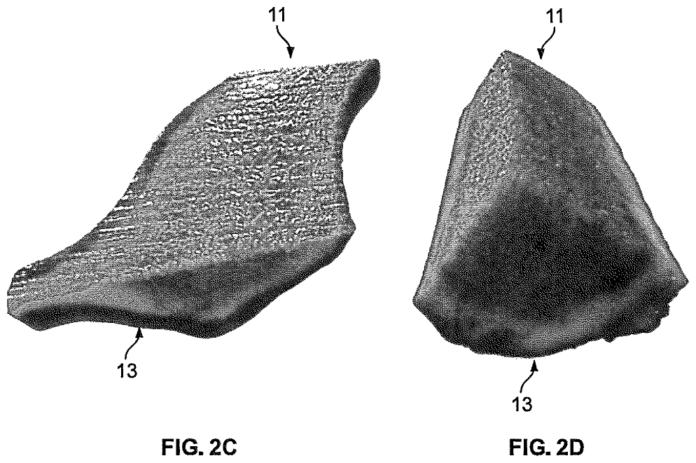

FIGS. 2A-2D show different views of sections of the inventive antler, where the denser outer portion of the antler is designated by the reference numeral 11 and the more porous lamellar bone that contains the additive is designated by the reference numeral 13. The sectional cuts are also exemplary and the cross sectional shape can vary from that shown in FIGS. 2A-2D.

The deer family antler can be from any member of the deer family, white tail deer, elk, moose, and the like.

As such, an invention has been disclosed in terms of preferred embodiments thereof which fulfills each and every one of the objects of the present invention as set forth above and provides a new and improved dog chew made from deer family antler and containing one or more additives designed to improve the health of the dog.

Of course, various changes, modifications and alterations from the teachings of the present invention may be contemplated by those skilled in the art without departing from the intended spirit and scope thereof. It is intended that the present invention only be limited by the terms of the appended claims.

We claim:

1. In a deer family antler pet chew, the improvement comprising one or more additives present in an effective amount in the deer family antler chew to improve the health of the ingesting the deer family antler pet chew.

2. The chew of claim 1, wherein the one or more additives are selected from the group consisting of vitamins, minerals, supplements for joints, for example, glucosamine and chondroitin, omega-3, -6 and -9 fish oils, linoleic acid, probiotics and prebiotics, for example, fructooligosaccharide and Bacillus Coagulans, natural ingredients as one or more of kelp, alfalfa herb, flaxseed meal, brewer's yeast, lecithin, spinach, garlic, chamomile, oatstraw, dandelion root, thyme, rosemary, peppermint, fennel, rose hips, licorice, ginger, barley grass, wheat grass, irish moss, chlorella and natural spirulina, digestive enzymes, and anti-oxidants.

3. The chew of claim 1, wherein the one or more additives are contained in at least the lamellar bone of the deer family antler.

4. The chew of claim 2, wherein the vitamins comprise one or more of Vitamin A, B vitamins (biotin, folate, niacin, pantothenic acid, riboflavin, thiamine, vitamin B-6, and vitamin B-12), Vitamin C, Vitamin D, Vitamin E, Vitamin K, and Choline.

5. The chew of claim 2, wherein the minerals comprise one or more of Calcium, Magnesium, Iron, Phosphorus, Potassium, Manganese, Chromium, Zinc, Copper, and Selenium.

6. A method of improving the health of a pet by providing the deer family antler pet chew of claim 1 to the pet for ingestion and health benefits from the one or more additives in the deer family antler pet chew.

7. The method of claim 6, wherein the pet is a dog.

8. A method of adding one or more additives to a deer family antler pet chew to improve at least the health of a pet ingesting the deer family antler pet chew, the method comprising the steps of:
a) providing a deer family antler pet chew;
b) providing a solution containing the one or more additives;
c) placing the deer family antler pet chew in the solution so that the one or more additives are retained in at least the lamellar bone of the deer family antler so as to provide a health benefit to a pet using the deer family antler pet chew.
